(19) United States
(12) Patent Application Publication
(10) Pub. No.: US 2026/0108328 A1
(43) Pub. Date: Apr. 23, 2026

(54) IMPLANT-SUPPORTED FULL ARCH PROSTHESIS

(71) Applicant: Digital Dental Fusion, Inc., Seoul (KR)

(72) Inventor: Daniel Hyunjae CHO, Las Vegas, NV (US)

(21) Appl. No.: 18/921,015

(22) Filed: Oct. 21, 2024

(51) Int. Cl. A61C 8/00; A61C 13/083; A61L 27/04; A61L 27/06; A61L 27/18; A61L 27/36

(52) U.S. Cl. CPC A61C 8/0095; A61C 13/083; A61L 27/047; A61L 27/06; A61L 27/18; A61L 27/3616; A61L 2400/18; A61L 2430/12

(57) ABSTRACT

Described herein is an implant-supported full arch prosthesis including a zirconia shell, a titanium bar inserted into the zirconia shell, and an inner cushioning liner formed in a space defined between the zirconia shell and the titanium bar, wherein connecting portions of the zirconia shell, the titanium bar, and the inner cushioning liner are surface treated with plasma. The implant-supported full arch prosthesis is lighter in weight than a conventional zirconia prosthesis and is capable of reducing impact directly applied to an implant during mastication.

### BACKGROUND

### Field of the Invention

Various embodiments of the present invention relate to an implant-supported full arch prosthesis, and more specifically, relate to an implant-supported full arch prosthesis provided to perform All-on-X dental implant procedures.

### Description of the Related Art

Dentures are artificial teeth that are attachable and detachable to the gums of a user for usage thereof. Dentures have an advantage in that a medical treatment period is short, and medical treatment costs are low. However, since dentures are not fixed, dentures may have weak masticatory force (chewing ability) during the process of mastication (chewing) in the mouth and may be readily separated from the gums when a user eats sticky foods. In addition, since dentures are supported only by the gums, dentures may cause pain or inflammation of the gums when used for a long period of time. Further, since dentures become loose due to absorption of the alveolar bone, the longer a user uses dentures, the more uncomfortable the user may feel during the process of mastication (chewing food) in the mouth.

Implant surgery solves the above-described problems related to dentures. Implant surgery is a treatment performed in such a manner that a support made of titanium is fixed to the alveolar bone of a missing tooth area so as to create a tooth root, and then a dental prosthesis is created on top of the tooth root. When a user chews food using implants, masticatory force of the implants is five to seven times stronger than that of dentures, and the implants may also prevent absorption of the alveolar bone. However, implant surgery requires a long treatment period and is expensive. Accordingly, in cases of patients having complete or partial edentulous (tooth loss), the burden on the patients for implant surgery is inevitably increased. Additionally, the number of supports fixed to the alveolar bone must be made equal to the number of missing teeth, which may cause damage to the gums and the alveolar bone. For these reasons, it is difficult to perform implant surgery on the elderly who have weak teeth.

Currently, a monolithic zirconia prosthesis, which is made entirely of zirconia, dominates more than 95% of the implant-supported full arch prosthesis market. The reason for this is that, since a zirconia material itself has high durability and strength, a prosthesis formed of zirconia is less likely to fall out or fracture than prostheses formed of other materials. Additionally, since the color of zirconia is similar to that of the teeth, zirconia may improve aesthetics of the monolithic zirconia prosthesis. Furthermore, it is difficult for plaque to be deposited on zirconia, so there is a low risk of inflammation. Moreover, since zirconia, unlike metal, does not cause an allergic reaction, it may be used with high biocompatibility.

However, the monolithic zirconia prosthesis has the following problems. First, solid zirconia has a specific gravity of about 6, which is heavier than titanium having a specific gravity of 4.5. For this reason, a user may feel uncomfortable when using the monolithic zirconia prosthesis. Second, solid zirconia is a hard material that does not absorb impact. Thus, when a user chews food, impact is directly applied to dental implants implanted into the alveolar bone. Therefore, as time passes, the dental implants implanted into the alveolar bone may fall out, or the alveolar bone having low density may be damaged. Third, due to high hardness and heavy weight of solid zirconia, when a user chews food, a noise caused by collision of the upper and lower teeth is so loud that the user may have a headache due to annoying noise around the entire head. Therefore, a user may feel very uncomfortable when using the monolithic zirconia prosthesis. Fourth, since solid zirconia is mainly manufactured using a milling machine instead of using a 3D printing technique, it may be difficult to reliably reproduce the original shape of the teeth and gums. Fifth, since an individual connecting portion such as a titanium base (a Ti-base) is manually bonded to another portion, precise bonding therebetween may not be achieved. In this case, a dental implant implanted into the alveolar bone may fall out.

The reason why the monolithic zirconia prosthetic is popular despite the above-described inconveniences is that it is better to tolerate the above-described side effects than to experience breakage or fracture of the prosthesis. In other words, when breakage or fracture of the prosthesis occurs, multiple dental procedures should be repeatedly performed to make a new prosthesis, which takes time and costs a lot of money. Zirconia has higher hardness than that of teeth. Accordingly, while a user chews food, impact may be applied to a treatment area of the user. On the other hand, due to high hardness of zirconia, a prosthesis formed of zirconia is less likely to cause breakage or fracture thereof than prostheses formed of other materials such as resin.

In order to solve the above-described problems of the monolithic zirconia prosthesis, an inventor of the present invention provided a “METHOD OF MANUFACTURING FIXED DENTURES USING PEEK AND ZIRCONIA AND FIXED DENTURES MANUFACTURED THEREFROM”, which was disclosed in Korean Patent No. 10-2085336 (Registration date: February 28, 2020). An object of Korean Patent No. 10-2085336 is to maximally reduce impact applied to an implant by forming a gum portion from medical polyetheretherketone (PEEK), thereby enabling a user to use the implant for a long period of time. However, when this invention is actually implemented, impact caused by mastication is directly applied to PEEK to which an implant is coupled. As described above, this invention has a problem in that it is difficult to maximally reduce the impact applied to the implant during mastication.

### SUMMARY

Therefore, one or more embodiments of the present invention have been made in view of the above problems, and one or more embodiments of the present invention provide an implant-supported full arch prosthesis that is formed of a zirconia material and has a light weight overall.

One or more embodiments may provide an implant-supported full arch prosthesis used for medical implant procedures in which multiple implants are implanted into the alveolar bone of a patient who has a completely or partially edentulous teeth, a frame is connected to the upper portions of the implants, and a prosthesis is fixed to the implants.

An implant-supported full arch prosthesis according to one or more embodiments of the present invention may compensate for the shortcomings of the above-described dentures and implants in the prior art and may take advantage of advantages of the dentures and implants. The implant-supported full arch prosthesis in one or more embodiments may be provided to fix the entire prosthesis with a minimum number of implants. Further, in the implant-supported full arch prosthesis, a frame may be connected to the upper portions of the implants so as to fix the prosthesis. In this case, since fewer implants are implanted into the alveolar bone than the number of missing teeth, it is possible to reduce damage to the gums, to shorten the time required for medical treatment, and to reduce costs for medical treatment. In addition, since the implant-supported full arch prosthesis is fixedly provided, masticatory force of the implant-supported full arch prosthesis is much stronger than that of dentures.

One or more embodiments of the present invention may provide an implant-supported full arch prosthesis that is formed of a medical dental filling material and a zirconia material and is capable of easily absorbing impact between the teeth of upper and lower jaws during the process of mastication (chewing) in the mouth.

One or more embodiments of the present invention may provide an implant-supported full arch prosthesis that is precisely processed by three-dimensional printing (3D Printing).

In accordance with one or more embodiments of the present invention, there may be provided an implant-supported full arch prosthesis formed to have a thin frame shape with a constant thickness and configured to provide a zirconia shell having a hollow formed therein, wherein the implant-supported full arch prosthesis is made of a zirconia material and is capable of reducing the weight thereof.

In one or more embodiments, the implant-supported full arch prosthesis may include a titanium bar made of a titanium material and formed to have a constant thickness, wherein the titanium bar may have a plurality of implant coupling holes formed therein and coupled to respective implants, and wherein the titanium bar may be inserted into the zirconia shell so as to prevent impact directly applied to the implants during mastication.

In one or more embodiments, the implant-supported full arch prosthesis may include an inner cushioning liner formed by filling, with a dental cushioning material, an empty space defined between the zirconia shell and the titanium bar inserted into the zirconia shell so as to absorb impact directly applied to the implants during mastication.

In one or more embodiments, plasma may be applied to connection portions of the zirconia shell, the titanium bar, the inner cushioning liner, and the implant coupling holes so as to perform surface treatment thereon, thereby removing surface impurities and increasing surface energy.

One or more embodiments may provide an implant-supported full arch prosthesis including a zirconia shell forming an entire outer surface of the implant-supported full arch prosthesis and having a first hollow formed therein, and having a uniform thickness according to a shape of gums and teeth of a patient; a titanium bar inserted into the first hollow formed in the zirconia shell so as to form a second hollow between the zirconia shell and the titanium bar, the second hollow having a gap defined between an inner surface of the zirconia shell and an outer surface of the titanium bar; and an inner cushioning liner formed by filling, with a cushioning material, the second hollow between the zirconia shell and the titanium bar, wherein connecting portions of the zirconia shell, the titanium bar, and the inner cushioning liner are surface treated with plasma.

In one or more embodiments, the titanium bar is formed to have a plurality of implant coupling holes configured to allow implants to be respectively coupled thereto.

In one or more embodiments, the gap defined between the inner surface of the zirconia shell and the outer surface of the titanium bar has a uniform distance.

In one or more embodiments, the zirconia shell has a thickness of about 1.3 mm.

In one or more embodiments, the titanium bar has a thickness of about 1.5 mm or more.

In one or more embodiments, the inner cushioning liner has a thickness in a range of 0.5 to 1.0 mm.

In one or more embodiments, the cushioning material of the inner cushioning liner is formed of polyetheretherketone.

### BRIEF DESCRIPTION OF THE DRAWINGS

The above and other objects, features, and other advantages of the present invention will be more clearly understood from the following detailed description taken in conjunction with the accompanying drawings, in which:

FIG. 1 is a drawing showing the internal structure of an implant-supported full arch prosthesis according to an embodiment of the present invention.

### DETAILED DESCRIPTION

Prior to description of the present invention in the present disclosure, specific structural or functional descriptions given in connection with embodiments of the present disclosure are merely illustrative, and embodiments of the present invention may be implemented in various forms. Further, it will be understood that the present disclosure is not intended to limit the present invention to embodiments described in the present disclosure.

Embodiments of the present invention may be modified in various ways and may have various embodiments, and specific embodiments are illustrated in the drawings in detail in the detailed description. However, it should be understood that the present invention is not limited to the specific embodiments, and the specific embodiments include all modifications, equivalents, and substitutes that fall within the spirit and technical scope of the present invention.

In embodiments of the present disclosure, the terms “uniform” and “constant” are interchangeable.

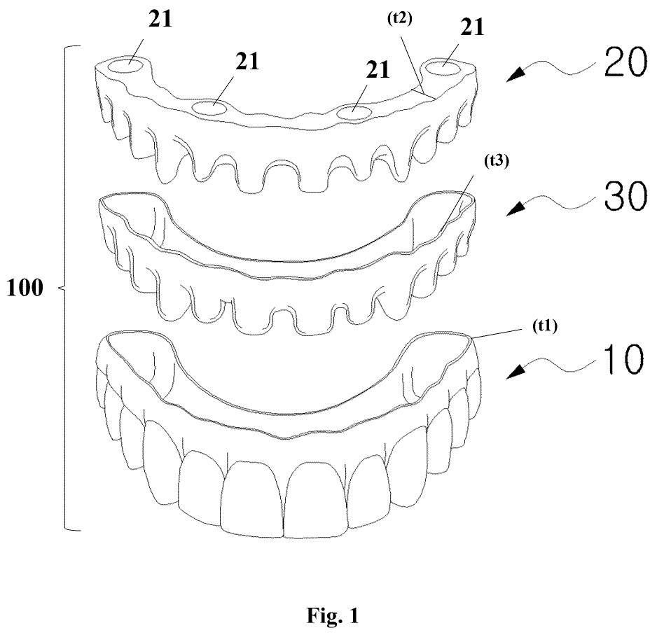

One or more embodiments of the present invention relate to an implant-supported full arch prosthesis 100 as shown in FIG. 1.

One or more embodiments of the present invention relate to an implant-supported full arch prosthesis 100 formed of a zirconia shell 10, a titanium bar 20 a plurality of implant coupling holes 21, and an inner cushioning liner. The zirconia shell 10 has a thickness denoted by t1. The titanium bar 20 has a thickness denoted by t2, and the inner cushioning liner 30 has a thickness denoted by t3.

A zirconia shell 10 shown in FIG. 1 is described below.

The zirconia shell 10 is formed of a zirconia material capable of being processed by three-dimensional printing (3D printing), and forms the outer surface of the entire prosthesis. The zirconia shell 10 is formed to have, by 3D printing, a thin frame shape with a constant thickness t1 in accordance with data related to the shape of the gums and the teeth of a patient, and has a hollow formed therein.

The outer surface of the frame of the zirconia shell 10 is formed to be identical to the shape of the gums and the teeth of a patient. In addition, the inner surface of the zirconia shell 10 is formed to extend along the outer surface of the frame of the zirconia shell 10 with a constant thickness t1. That is, the zirconia shell 10 is characterized in that a thickness of the outer surface and a thickness of the inner surface are uniformly formed.

When the thickness of the outer surface of the frame of the zirconia shell 10 and the thickness of the inner surface thereof are not uniformly formed, the weight of the zirconia shell 10 varies depending on the location of the zirconia shell 10, leading to an imbalance in weight of the zirconia shell. Accordingly, the imbalance in weight may not only cause a fracture of the zirconia shell, but also lead to deterioration in wearing comfort of the implant-supported full arch prosthesis 100.

When the thickness of the zirconia shell 10 is too large, the zirconia shell becomes heavy such that there is no significant difference in wearing comfort from conventional solid zirconia. On the other hand, when the thickness of the zirconia shell is too small, it may be difficult to obtain a uniform thickness of the zirconia shell by 3D printing, and a fracture of the zirconia shell may easily occur due to insufficient thickness. Accordingly, in consideration of the aforementioned thickness relationship, the thickness t1 of the zirconia shell is preferably 1.2 mm to 1.3 mm, most preferably 1.3 mm.

A titanium bar 20 shown in FIG. 1 is described below.

The titanium bar 20 of one or more embodiments is inserted into the zirconia shell 10 so as to connect implants to the implant-supported full arch prosthesis 100.

The titanium bar 20 of one or more embodiments is inserted into an inner hollow formed in the zirconia shell 10 such that a hollow having a constant thickness is formed between the titanium bar 20 and the zirconia shell 10 in proportion to the inner surface of the zirconia shell 10 defining a gap between the inner surface of the zirconia shell 10 and the outer surface of the titanium bar 20, in which the gap has a uniform distance therebetween.

That is, the outer surface of the titanium bar needs to be formed in proportion to the inner surface of the zirconia shell so as to be spaced apart from the inner surface of the frame of the zirconia shell at a constant interval therebetween. When the outer surface of the titanium bar and the inner surface of the frame of the zirconia shell are not spaced apart from each other with a constant interval therebetween, the weight of the titanium bar varies depending on the location thereof, which causes an imbalance in weight. Further, this imbalance may not only cause a fracture of the titanium bar, but also lead to deterioration in wearing comfort of the implant-supported full arch prosthesis 100.

When the thickness of the titanium bar is too small, the titanium bar may not appropriately perform a bridge function of connecting the implants to the implant-supported full arch prosthesis 100. Accordingly, the titanium bar needs to constantly maintain an appropriate thickness t2 so as to perform the bridge function.

Specifically, in consideration of the aforementioned thickness characteristic, the thickness t2 of the titanium bar is most preferably 1.5 mm or more.

The titanium bar may further include a plurality of implant coupling holes 21 to be respectively coupled to the implants.

An inner cushioning liner 30 of the present invention will be described.

The inner cushioning liner 30 is formed by filling, with a dental cushioning material, an empty space defined between the zirconia shell 10 and the titanium bar 20 inserted into the zirconia shell. Accordingly, the inner cushioning liner may have a function of absorbing impact between the teeth of upper and lower jaws during the process of mastication (chewing).

The cushioning material of the inner cushioning liner 30 may be formed of polyetheretherketone. Polyetheretherketone is also referred to as PEEK and is a high-performance thermoplastic polymer having various properties such as high strength, high rigidity, high temperature resistance, chemical resistance, and low smoke emission. Since polyetheretherketone is lighter in weight than metal materials such as zirconia and titanium, the same may play a role in compensating for the shortcomings of a conventional implant-supported full arch prosthesis that is made only using zirconia or titanium and has a heavy weight. In addition, polyetheretherketone has elastic properties different from metal materials. Accordingly, polyetheretherketone may perform a cushioning function of absorbing impact directly applied to dentures or implants during mastication.

In addition, the inner cushioning liner may be formed of other cushioning materials that are lighter in weight than zirconia or titanium and have elastic properties different from metal materials. For example, the inner cushioning liner may be formed of ULTEM, nylon, polytetrafluoroethylene (PTFE), PPSU, acrylic resin, and the like.

The inner cushioning liner needs to be filled with polyetheretherketone so that the outer surface of the titanium bar and the inner surface of the frame of the zirconia shell are proportionally spaced apart from each other with a constant interval therebetween.

When the outer surface of the titanium bar and the inner surface of the frame of the zirconia shell are not evenly spaced apart from each other, the weight of the titanium bar varies depending on the location of the titanium bar, which causes an imbalance in weight. Further, this imbalance may not only cause a fracture of the titanium bar, but also lead to deterioration in wearing comfort of the implant-supported full arch prosthesis 100.

When the thickness of the inner cushioning liner is too large, the thickness of the zirconia shell or the titanium bar becomes small, which leads to deterioration in hardness of the zirconia shell or the titanium bar. On the other hand, when the thickness of the inner cushioning liner is too small, the thickness of the zirconia shell or the titanium bar becomes large, which causes an increase in weight of the zirconia shell or the titanium bar. As a result, it may be difficult to achieve an advantage of the present invention. Therefore, it is required to constantly maintain an appropriate thickness t3 of the inner cushioning liner.

Specifically, in consideration of the aforementioned thickness relationship, the thickness t3 of the inner cushioning liner is preferably 0.5 to 1.0 mm, most preferably 0.5 mm.

The implant-supported full arch prosthesis 100 of the present invention is characterized in that plasma is applied to connecting portions of the zirconia shell, the inner cushioning liner, and the titanium bar so as to perform surface treatment thereon. Through the above-mentioned plasma treatment, surface impurities are removed, and surface energy is increased, thereby making it possible to improve biocompatibility of the implants.

As is apparent from the above description, the present invention provides the following effects and/or advantages.

Unlike a heavy conventional monolithic zirconia prosthesis, the inside of which is fully filled with materials, an implant-supported full arch prosthesis 100 of the present invention is formed of a thin zirconia shell having a hollow portion formed therein and, as such, the total weight of the implant-supported full arch prosthesis 100 is reduced by about 50%, thereby having an effect of realizing a light weight close to the weight of the natural teeth and increasing wearing comfort of the implant-supported full arch prosthesis 100.

Further, according to the present invention, since a titanium bar connected to an implant is inserted into a zirconia shell, impact between the teeth of upper and lower jaws during the process of mastication is not directly applied to the implant, thereby having an effect of extending the period of use by reducing damage to the implant.

Additionally, unlike a conventional prosthesis using individual connecting devices, in the implant-supported full arch prosthesis 100 of the present invention, a personalized titanium bar is ultra-precisely formed, and then the titanium bar is inserted into the prosthesis, thereby having an effect of not only increasing suitability of the prosthesis, but also increasing strength of the prosthesis by supporting the zirconia shell.

Furthermore, according to the present invention, an inner cushioning liner fills a space defined between the zirconia shell having high hardness and the titanium bar, thereby enabling the inner cushioning liner to serve as a cushioning portion configured to reduce impact directly applied to the gums during the process of mastication. In this manner, it is possible to achieve an effect of improving wearing comfort of the implant-supported full arch prosthesis 100.

In addition, in the case of a monolithic zirconia prosthesis having a sold interior without a hollow formed therein, when the monolithic zirconia prosthesis is sintered at 1,600° C., internal tension that is difficult to identify with the naked eye occurs. Accordingly, when the monolithic zirconia prosthesis is contracted by about 20%, a fracture may occur. However, the implant-supported full arch prosthesis 100 of the present invention is formed to have a thin thickness including a hollow, thereby reducing occurrence of such a fracture.

Moreover, while the conventional monolithic zirconia prosthesis makes an annoying frictional sound, the implant-supported full arch prosthesis 100 of the present invention makes a natural sound such as a sound made when the upper and lower teeth contact each other, thereby having an effect of reducing psychological trauma of a medical operator.

Further, the present invention has an effect of naturally reproducing, when the zirconia shell is produced, the shape of the teeth and gums close to the original shape by using 3D printing.

Additionally, according to one or more embodiments of the present invention, plasma is applied to connecting portions of the zirconia shell, the inner cushioning liner, and the titanium bar so as to perform surface treatment thereon, thereby improving biocompatibility of the implant and providing a fast and stable osseointegration process. Accordingly, it is possible to achieve an effect of increasing the success rate and quality of implant surgery.

1. An implant-supported full arch prosthesis, comprising:
a zirconia shell forming an entire outer surface of the implant-supported full arch prosthesis and having a first hollow defined therein, and having a uniform thickness according to a shape of gums and teeth of a patient;
a titanium bar inserted into the first hollow defined in the zirconia shell so as to form a second hollow between the zirconia shell and the titanium bar, the second hollow having a gap defined between an inner surface of the zirconia shell and an outer surface of the titanium bar; and
an inner cushioning liner disposed in the second hollow between the zirconia shell and the titanium bar, the inner cushioning liner including a cushioning material,
wherein connecting portions of the zirconia shell, the titanium bar, and the inner cushioning liner are surface treated with plasma,
wherein the inner cushioning liner has a thickness in a range of 0.5 to 1.0 mm,
wherein the zirconia shell has a thickness of 1.3 mm, and
wherein the titanium bar has a thickness of about 1.5 mm.

2. The implant-supported full arch prosthesis according to claim 1, wherein the titanium bar includes a plurality of implant coupling holes configured to allow implants to be respectively coupled thereto.

3. The implant-supported full arch prosthesis according to claim 1, wherein the gap defined between the inner surface of the zirconia shell and the outer surface of the titanium bar has a uniform distance.

4. (canceled)

5. (canceled)

6. (canceled)

7. The implant-supported full arch prosthesis according to claim 1, wherein the cushioning material of the inner cushioning liner is composed of polyetheretherketone.
